(19) United States
(12) Patent Application Publication
(10) Pub. No.: US 2011/0053268 A1
(43) Pub. Date: Mar. 3, 2011

(54) METHOD FOR DIAGNOSIS OF ABNORMAL IRON METABOLISM USING ACTIVE HEPCIDIN AS INDICATOR

(76) Inventor: Naohisa Tomosugi, Ishikawa (JP)

(21) Appl. No.: 12/311,842

(22) PCT Filed: Mar. 16, 2007
(86) PCT No.: PCT/JP2007/055361

§ 371 Date: Oct. 5, 2010

(30) Foreign Application Priority Data
Oct. 17, 2006 (JP) 2006-282298

(51) Int. Cl. C12N 5/0789; G01N 33/50; C07K 14/575; C07K 7/08; C07K 16/18; H01J 49/00; G01N 33/68

(52) U.S. Cl. 435/372; 436/86; 530/326; 530/325; 530/324; 530/387.3; 250/282; 73/61.41

(57) ABSTRACT

The present inventor employed surface enhanced laser desorption/ionization time-of-flight mass spectrometry (SELDI-TOF-MS) to analyze distinctive serum proteomic patterns of hemodialysis patients. The present inventor found three peptides at 2,192, 2,789, and 2,851 m/z that showed a significant correlation with the levels of serum ferritin. The molecular sizes of the 2,192 and 2,789 m/z matched well with the reported sizes of hepcidin-20 and -25, respectively, and the latter completely coincided with the size of synthetic hepcidin-25. It would be possible to diagnose abnormal iron metabolism by detecting bioactive forms of hepcidin in the serum.

### TECHNICAL FIELD

The present invention relates to methods for the diagnosis of abnormal iron metabolism using active hepcidin as an indicator.

### BACKGROUND ART

The current understanding of the regulation of iron metabolism is based on the biology of a number of critical proteins, which include transferrin, transferrin receptors, ferritin, iron regulatory proteins, divalent metal transporter 1, ferroportin, and hepcidin (Non-patent Documents 1 to 4). Among these factors, plasma transferrin and ferritin are generally measured in a laboratory as an indicator of the total iron binding capacity and overall iron storage, respectively. The peptide hepcidin, which is produced by the liver, controls plasma iron levels by regulating the absorption of food iron from the intestine as well as the release of iron from macrophages. Furthermore, hepcidin is also an acute-phase reactant with antimicrobial activity induced by inflammation (Non-patent Documents 5 to 7). Most studies that confirm the role of hepcidin in iron metabolism, inflammation, anemia, and hypoxia were performed in vitro or using experimental mice (Non-patent Documents 5 and 8); therefore, its role in human diseases is unclear.

Renal anemia in hemodialysis patients can be clinically alleviated by the administration of human recombinant erythropoietin (Non-patent Document 9); however, the exact mechanism of iron metabolism in these patients is largely unknown. In several clinical studies, the amount of hepcidin was estimated based on the levels of prohepcidin in urine (Non-patent Documents 6 to 10 and 13) or serum (Non-patent Documents 14 to 16) or based on the levels of mRNA expression in the liver (Non-patent Document 10); however, it has been reported that serum prohepcidin concentration does not clearly correlate with any red cell indices or with iron status (Non-patent Document 14) and that the concentration of prohepcidin may increase because of its accumulation. In addition, prohepcidin, which is measured by enzyme-linked immunosorbent assay (ELISA), is not bioactive and no specific function has been identified. Hepcidin-20, -22, and -25, which are cleaved from prohepcidin by convertases, are the active forms of prohepcidin (Non-patent Document 6); however, there are few reports on the quantitative evaluation of the bioactive forms of hepcidin, primarily because of difficulties involved in the development of specific antibodies against these bioactive forms of hepcidin, which have compact folded structures (Non-patent Document 17).

Hepcidin, which is a key regulator of iron metabolism, is expressed in the liver, distributed in blood, and excreted in urine. To date, some diagnostic methods using hepcidin as an indicator have been reported (Patent Documents 1 to 4); however, no reliable and practical method for the measurement of the bioactive forms of hepcidin in serum has been developed.

The SELDI-based ProteinChip System® (Ciphergen Biosystems, Inc., Fremont, Calif., USA) array technology has been successfully used to detect relevant biomarkers in a wide variety of diseases, which include immunological conditions, cancer, neurological conditions, cardiovascular conditions, and diseases of the lacrimal gland (Non-patent Documents 18 to 21). SELDI technology is based on a classical solid-phase extraction chromatography method combined with direct laser desorption/ionization mass spectrometric detection and requires minimal amounts of biological fluid, without pretreatment. This technology enables the evaluation of the subtle differences between disease and control states in the expression of individual proteins or groups of proteins in various fluids, which include serum, urine, tears, and cerebrospinal fluid. Furthermore, it has a number of advantages, which include high-throughput capability, very high sensitivity for the detection of proteins in the picomole to femtomole ranges, high resolution for low-molecular-weight proteins (i.e., below 20 kDa), and facility of operation.

Documents of related prior arts for the present invention are described below.

### Non-patent Document 1: Andrews N C. Best Pract Res Clin Haematol. 2005; 18:159-169.

### Non-patent Document 2: Philpott C C. Hepatology. 2002; 35:993-1001.

### Non-patent Document 3: Fleming R E. Curr Opin Gastroenterol. 2005; 21:201-206.

### Non-patent Document 4: Ganz T. Blood. 2003; 102:783-788.

Non-patent Document 5: Pigeon C, Ilyin G; Courselaud B, et al. J Biol. Chem. 2001 16; 276:7811-7819.
Non-patent Document 6: Park C H, Valore E V, Waring A J, Ganz T. J Biol. Chem. 2001; 276:7806-7810.
Non-patent Document 7: Krause A, Neitz S, Magert H J, et al. FEBS Lett. 2000; 480:147-150.
Non-patent Document 8: Nicolas G, Chauvet C, Viatte L, et al. J Clin Invest. 2002; 110:1037-1044.
Non-patent Document 9: National Kidney Foundation. Am J Kidney Dis. 1997; 30(Suppl 3):S192-240.
Non-patent Document 10: Detivaud L, Nemeth E, Boudjema K, et al. Blood. 2005; 106:746-748.
Non-patent Document 11: Kemna E, Tjalsma H, Laarakkers C, Nemeth E, Willems H, Swinkels D. Blood. 2005 Jul. 19; [Epub ahead of print]
Non-patent Document 12: Nemeth E, Rivera S, Gabayan V, et al. J Clin Invest. 2004; 113:1271-1276.

### Non-patent Document 13: Nemeth E, Valore E V, Territo M, Schiller G, Lichtenstein A, Ganz T. Blood. 2003; 101:2461-2463.

### Non-patent Document 14: Taes Y E, Wuyts B, Boelaert J R, De Vriese A S, Delanghe J R. Clin Chem Lab Med. 2004; 42:387-389.

Non-patent Document 15: Kulaksiz H, Gehrke S G, Janetzko A, et al. Gut. 2004 May; 53(5):735-743.

### Non-patent Document 16: Dallalio G, Fleury T, Means R T. Br J. Haematol. 2003; 122:996-1000.

Non-patent Document 17: Hunter H N, Fulton D B, Ganz T, Vogel H J. J Biol. Chem. 2002; 277:37597-37603.
Non-patent Document 18: Petricoin E F, Ardekani A M, Hitt B A, et al. Lancet. 2002; 359:572-577.
Non-patent Document 19: Sanchez J C, Guillaume E, Lescuyer P, et al. Proteomics. 2004; 4:2229-2233.

### Non-patent Document 20: Stanley B A, Gundry R L, Cotter R J, Van Eyk J E. Dis Markers. 2004; 20:167-178.

### Non-patent Document 21: Tomosugi N, Kitagawa K, Takahashi N, Sugai S, Ishikawa I. J Proteome Res. 2005; 4:820-825.

Patent Document 1: Japanese Patent Application Kokai Publication No. (JP-A) 2005-134387 (unexamined, published Japanese patent application)

### Patent Document 2: US 2004/0096987 A1

### Patent Document 3: US 2004/0096990 A1

### Patent Document 4: US 2006/0019339 A1

### DISCLOSURE OF THE INVENTION

### Problems to be Solved by the Invention

An objective of the present invention is to discover marker proteins for abnormal iron metabolism and to provide inventions using the marker proteins. More specifically, the objective of the present invention is to provide methods for the diagnosis of abnormal iron metabolism that use active hepcidin as an indicator; methods for the diagnosis of diseases that cause abnormal iron metabolism that use active hepcidin as an indicator; methods for the selection of optimal agents for the treatment of subjects affected with such a disease; methods for the determination of the timing to administer agents that are used to treat the diseases; methods for the assessment of the restoration of bone marrow function; and kits and compositions that are used in these methods. Another objective of the present invention is to provide novel uses of agents that are used to prevent or treat diseases that cause abnormal iron metabolism.

### Means for Solving the Problems

The present inventor employed surface-enhanced laser desorption/ionization time-of-flight mass spectrometry (SELDI-TOF-MS) to analyze distinctive serum proteomic patterns of hemodialysis patients. Among the 114 peaks observed in the range of 1,000 to 15,000 m/z, the present inventor found three peptides at 2,192, 2,789, and 2,851 m/z that showed a significant correlation with the levels of serum ferritin. The molecular sizes of two of these, 2,192 and 2,789 m/z, matched well with the reported sizes of hepcidin-20 and -25, respectively, and the latter completely coincided with the size of synthetic hepcidin-25. Furthermore, these two peptides were strongly cationic and had four internal disulfide bridges; this provided evidence that they were bioactive forms of hepcidin. The serum levels of these peptides increased during iron administration, decreased as a result of repeated bleeding, and were elevated in a patient with acute urinary infection; thus, it would be possible to diagnose abnormal iron metabolism by detecting bioactive forms of hepcidin in serum.

Specifically, the present invention provides:

[1] a method for diagnosing an abnormal iron metabolism, which comprises the step of determining the amount of active hepcidin in a serum sample prepared from blood collected from a subject;
[2] a method for diagnosing a disease that causes an abnormal iron metabolism, which comprises the step of determining the amount of active hepcidin in a serum sample prepared from blood collected from a subject;
[3] a method for selecting an optimal agent for treating a subject affected with a disease that causes an abnormal iron metabolism, which comprises the steps of:

(a) determining the amount of active hepcidin in a serum sample prepared from blood collected from a subject administered with a test agent;

(b) processing, in the same way as in step (a), each of one or more test agents different from the test agent used in step (a);

(c) comparing the amount of each active hepcidin determined in step (a) and step (b) with that in a control serum sample; and

(d) selecting a test agent that brings the amount of active hepcidin closest to that in the control serum sample, based on the comparison result obtained in step (c);

[4] a method for determining the timing to administer an agent used to treat a disease which causes an abnormal iron metabolism, which comprises the step of determining the amount of active hepcidin in each serum sample prepared from blood collected from a subject over time;
[5] a method for assessing restoration of bone marrow function, which comprises the step of determining the amount of active hepcidin in each serum sample prepared from blood collected over time from a subject treated by radiotherapy or bone marrow transplantation;
[6] the method of any one of [1] to [4], wherein the abnormal iron metabolism is mediated by IL-6;
[7] the method of [6], wherein the IL-6-mediated abnormal iron metabolism is anemia in Castleman's disease;
[8] the method of any one of [1] to [4], wherein the abnormal iron metabolism is caused by a bone marrow dysfunction;
[9] the method of any one of [1] to [8], wherein the active hepcidin is hepcidin-20 and/or hepcidin-25;
[10] the method of any one of [1] to [8], wherein the step of determining the amount of active hepcidin in a serum sample comprises the steps of:

(a) mixing a serum sample with a carrier that has the property of binding to the active forms of (1) hepcidin-20 and/or (2) hepcidin-25; and

(b) determining the amount of active hepcidin bound to the carrier;

[11] the method of [10], wherein step (a) is conducted under conditions where only a polypeptide with a substantial pI value of 8 or more binds to the carrier;
[12] the method of any one of [1] to [11], wherein the active hepcidin is detected by SELDI-TOF-MS;
[13] a kit to be used in the method of any one of [1] to [12];
[14] an agent that is used to treat or prevent a disease that causes an abnormal iron metabolism wherein the agent is administered to a subject in whom the amount of active hepcidin in serum is increased or decreased when compared with a subject without a disease that causes the abnormal iron metabolism; and
[15] a composition used in the method of any one of [1] to [12], which comprises a polypeptide with a substantial pI value of 8 or more and is purified from blood collected from a subject.

### BRIEF DESCRIPTION OF THE DRAWINGS

FIG. 1 shows a representative serum profiling in the mass-to-charge ratio range of 2,000 to 4,000 m/z. Cases 1 and 2 were hemodialysis patients and cases 3 and 4 were volunteers with normal renal function. Peaks at 2,192, 2,789, and 2,851 m/z were observed in case 1 and not in cases 2 and 4. In case 3, the peak at 2,789 m/z was slightly increased.

FIG. 2 shows the correlation between the intensities of each peak and ferritin. There were significant correlations between the level of serum ferritin and the intensity of the 2,192 (A), 2,789 (B), or 2,851 m/z (C) peaks in hemodialysis patients or with the intensity of the 2,789 m/z (B) peak in the healthy volunteers with normal renal function (D).

FIG. 3 shows the characteristic evaluation of the peptides with peaks at 2,192 and 2,789 m/z. The evaluation of pI (A) was performed using cationic CM10 chips in the pH range of 8 to 10. Both peptides bound to the chip at pH 10. The peak at 2,789 m/z from isolated and purified serum (B, top) appeared as another peak at 2,797 m/z after reduction by DTT treatment (B, middle). The molecular weight increased by 8 Da after the reduction. Another 3,253 m/z peak appeared after alkylation by iodoacetamide treatment (B, bottom) and the molecular weight increased by 456 Da.

FIG. 4 shows the amino acid sequence analyzed by CID tandem MS after immobilization of purified 2,789 m/z onto the protein chip. Mascot search of the produced ions revealed that the amino acid sequence was identical to that of hepcidin-25.

FIG. 5 shows a calibration curve of the semi-quantitative measurement method for hepcidin-25. Synthetic hepcidin-25 was added at 4, 16, 64, or 256 nM to serum without the 2,789 m/z peak. A measurement using a protein chip showed that the correlation was r=0.9, while the CV value was 20 to 30%. This suggests a clinical applicability as a semi-quantitative measurement method.

FIG. 6 shows the clearance of serum peptides by hemodialysis. The intensities of peptides at 2,192 (A), 2,789 (B), and 2,851 m/z (C) were significantly reduced after hemodialysis. The removal rates of the 2,192, 2,789, and 2,851 m/z peptides by hemodialysis were 45±19%, 46±23%, and 52±30%, respectively.

FIG. 7 shows the effect of iron status on the 2,192 and 2,789 m/z peaks. Ferritin increased from 11.3 to 273 ng/ml in a patient administered with iron intravenously for eight weeks and the expression of the 2,192 and 2,789 peaks increased (A, C). The expression of the 2,192 and 2,789 peaks decreased in a patient whose ferritin was reduced from 202 to 28 ng/ml because of menstrual bleeding over a period of four months (B, D).

FIG. 8 shows changes in the peaks at 2,192, 2,436, and 2,789 m/z in a patient with pyelonephritis. Expression of the 2,192 and 2,789 peaks in urine samples (A) and serum samples (B) decreased after treatment with antibiotics for 17 days. The 2,436 m/z peak was observed exclusively in urine samples. The 2,192, 2,436, and 2,789 m/z peaks corresponded to hepcidin-20, -22, and -25, respectively.

FIG. 9 shows the correlation between the intensity of the peptide at 2,789 m/z and IL-6. The concentrations of serum IL-6 were lower than 20 pg/ml in all hemodialysis patients, with the exception of one patient with SLE. In hemodialysis patients (filled circles, n=40), there was no correlation between the 2,789 m/z peak and IL-6; however, in the patients with SLE, pyelonephritis, pneumonia, and sepsis (open circles, n=16), there was a good correlation between the 2,789 m/z peak and IL-6 (r=0.6).

FIG. 10 shows the time courses of hepcidin-25, ferritin, hemoglobin, and albumin before and after tocilizumab administration in case 1.

FIG. 11 shows the time courses of hepcidin-25, ferritin, hemoglobin, and albumin before and after tocilizumab administration in case 2.

FIG. 12 shows the time courses of urine hepcidin-25 and urine hepcidin-20 before and after tocilizumab administration in case 1.

FIG. 13 shows the time course of the reticulocyte count before and after SCT treatment. The recovery of the reticulocyte count was delayed in cases 1, 4, 6, and 10.

FIG. 14 shows the time course of IL-6 before and after SCT treatment. IL-6 reached a peak at week +1 in seven cases, which excluded cases 3, 4, and 6.

FIG. 15 shows the time course of serum hepcidin-25 before and after SCT treatment. The hepcidin level reached a peak at week +1 in eight cases, which excluded two cases; in one of these cases, the hepcidin level reached a peak at week 0 and in the other case the peak was reached at week +2. In cases 1, 4, 6, and 10, hepcidin remained at a significantly high level, even at week +4. The upper limit is 30 AU in healthy persons.

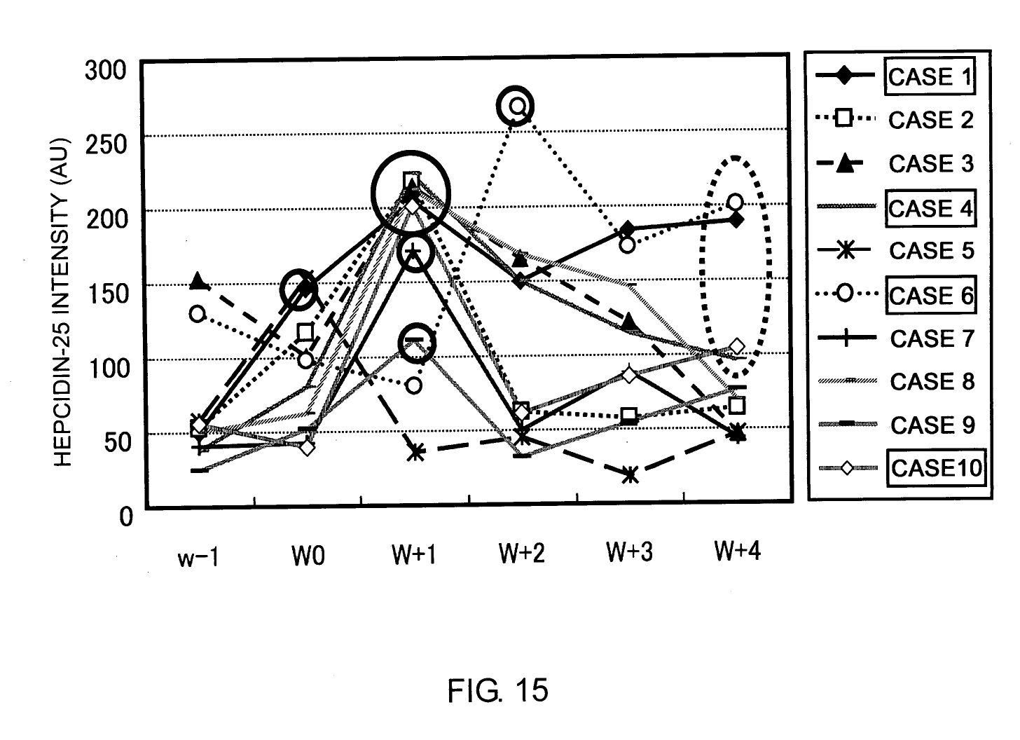

### MODE FOR CARRYING OUT THE INVENTION

The present invention provides methods for the diagnosis of abnormal iron metabolism, which comprise the step of determining the amount of active hepcidin in a serum sample prepared from blood collected from a subject.

In the diagnostic methods of the present invention, when the amount of active hepcidin in a serum sample prepared from blood collected from a subject is larger or smaller than that in a control serum sample, the subject is diagnosed as having abnormal iron metabolism. Furthermore, this diagnosis enables the assessment of whether a patient is affected with a disease that causes abnormal iron metabolism or has a high risk for such disease. Alternatively, the methods enable the diagnosis of abnormal iron metabolism caused by hematopoietic dysfunction of bone marrow, which results from administration of an anticancer agent or immunosuppressant, radiotherapy, bone marrow transplantation, etc. As the hematopoietic function of the bone marrow is higher, the amount of active hepcidin decreases. Conversely, as the hematopoietic function is lower, the amount of active hepcidin increases.

When a subject is human, in general, medical practitioners (including persons who are instructed by medical practitioners; the same is applied henceforth) diagnose such diseases. The data on the amount of active hepcidin, which are obtained by the diagnostic methods of the present invention, are useful in medical practitioners' diagnosis; thus, the performance of the diagnostic methods of the present invention has an aspect of collecting and providing data that are useful for medical practitioners to diagnose such diseases.

The present invention also provides methods for the selection of optimal agents for the treatment of subjects with a disease that causes abnormal iron metabolism, which comprise the steps of:

(a) determining the amount of active hepcidin in a serum sample prepared from blood collected from a subject administered with a test agent;

(b) processing, in the same way as in step (a), each of one or more test agents different from the test agent used in step (a);

(c) comparing the amount of each active hepcidin determined in step (a) and step (b) with that in a control serum sample; and

(d) selecting a test agent that makes the amount of active hepcidin closest to that in the control serum sample, based on the comparison result obtained in step (c).

When the subject is human, in general, medical practitioners select optimal agents for the treatment of such diseases. The data that are obtained using the methods of the present invention for the selection of agents are useful when medical practitioners select such agents; thus, the performance of the methods of the present invention for the selection of agents has an aspect of collecting and providing data that are useful for medical practitioners to select agents.

In the methods of the present invention for the selection of agents, each test agent is administered to the same subject. An optimal agent can be selected from different (two or more) test agents using the methods of the present invention for the selection of agents.

More specifically, the methods of the present invention for the selection of agents are methods for the selection of optimal agents to treat subjects with a disease that causes abnormal iron metabolism and whose active hepcidin has been increased, which comprise the steps of:

(1) determining the amount of active hepcidin in a serum sample prepared from blood collected from a subject administered with a test agent;

(2) processing, in the same way as in step (1), each of one or more test agents different from the test agent used in step (1);

(3) comparing the amount of each active hepcidin determined in step (1) and step (2) with that in a control serum sample; and

(4) selecting a test agent that reduces active hepcidin to an amount closest to that in the control serum sample, based on the comparison result obtained in step (3).

More specifically, the methods of the present invention for the selection of agents are methods for the selection of the optimal agents to treat subjects with a disease that causes abnormal iron metabolism and whose active hepcidin has been reduced, which comprise the steps of:

(1) determining the amount of active hepcidin in a serum sample prepared from blood collected from a subject administered with a test agent;

(2) processing, in the same way as in step (1), each of one or more test agents different from the test agent used in step (1);

(3) comparing the amount of each active hepcidin determined in step (1) and step (2) with that in a control serum sample; and

(4) selecting a test agent that increases active hepcidin to an amount closest to that in the control serum sample, based on the comparison result obtained in step (3).

Furthermore, the present invention provides methods for the determination of the timing to administer agents that are used to treat diseases that cause abnormal iron metabolism, which comprise the step of determining the amount of active hepcidin in each serum sample prepared from blood collected from a subject over time.

In the methods of the present invention for the determination of the timing to administer agents, when the determined amount of active hepcidin is increased or decreased when compared with that in the initial measurement, the time point is judged to be appropriate for administration of the agent.

When the subject is human, in general, medical practitioners determine the timing for administration of agents. The data that are obtained using the methods of the present invention for the determination of the timing to administer agents are useful when medical practitioners determine the timing for administration of agents; thus, the performance of the methods of the present invention for the determination of the timing to administer agents has an aspect of collecting and providing data that are useful for medical practitioners to determine the timing for administration of agents.

In the methods of the present invention for the determination of the timing to administer agents, the subjects include, for example, those who may be affected with a disease that causes abnormal iron metabolism.

In addition, the present invention provides methods for the determination of the dose of an agent that is used to treat diseases that cause abnormal iron metabolism, which comprise the step of determining the amount of active hepcidin in each serum sample prepared from blood collected from a subject over time.

In the methods of the present invention for the determination of the dose of an agent, when the determined amount of active hepcidin is increased or decreased as compared with that in the initial measurement, the dose is judged to be appropriate.

When the subject is human, in general, medical practitioners determine the dose of an agent. The data that are obtained using the methods of the present invention for the determination of the dose of an agent are useful when medical practitioners determine the dose of an agent; thus, the performance of the methods of the present invention for the determination of the dose of an agent has an aspect of collecting and providing data that are useful for medical practitioners to determine the dose of an agent.

In the methods of the present invention for the determination of the dose of an agent, the subjects include, for example, those who may be affected with a disease that causes abnormal iron metabolism.

In the methods of the present invention for the selection of the agents, methods for the determination of the timing to administer an agent, and methods for the determination of the dose of an agent, the test agent includes known agents for the treatment of diseases that cause abnormal iron metabolism. Such therapeutic agents include, for example, erythropoietin, anti-IL-6 agents, and iron preparations, but are not limited thereto.

The present invention also provides methods for assessment of the restoration of bone marrow function, which comprise the step of determining the amount of active hepcidin in each serum sample prepared from blood collected over time from subjects who have been treated by radiotherapy or bone marrow transplantation.

In the methods of the present invention for assessment of the restoration of bone marrow function, when the determined amount of active hepcidin is larger than the amount of active hepcidin in a serum sample prepared from blood collected from a subject with normal bone marrow function, the restoration is judged to be delayed; when the determined amount is comparable, the bone marrow function is judged to be restored.

When the subject is human, in general, medical practitioners assess the restoration of bone marrow function. The data that are obtained using the methods of the present invention for assessment of the restoration of bone marrow function are useful when medical practitioners assess the restoration of bone marrow function; thus, the performance of the methods of the present invention for assessment of the restoration of bone marrow function has an aspect of collecting and providing data that are useful for medical practitioners to assess the restoration of bone marrow function.

The methods of the present invention are conducted using serum samples prepared in medical centers. Alternatively, the methods of the present invention are conducted using blood collected in medical centers. In the latter case, persons who conduct the methods of the present invention can prepare serum samples from blood using methods known to those skilled in the art. For example, collected blood samples are centrifuged and the sera are collected. The resulting sera can be used as samples after dilution to an appropriate concentration, without special pretreatment.

In the present invention, the preferred subjects or targets are humans, rats, and mice, but are not limited thereto. The subjects or targets also include nonhuman animals; for example: monkeys, bovines, sheep, dogs, cats, and hamsters.

Herein, the “active hepcidin” refers to hepcidin-20 (polypeptide comprising the amino acid sequence of SEQ ID NO: 1) and/or hepcidin-25 (polypeptide comprising the amino acid sequence of SEQ ID NO: 2), for example, when the target or subject is a human. Alternatively, the “active hepcidin” herein refers to hepcidin (polypeptide comprising the amino acid sequence of SEQ ID NO: 3) when the target or subject is a rat, or to hepcidin-1 (polypeptide comprising the amino acid sequence of SEQ ID NO: 4) and/or hepcidin-2 (polypeptide comprising the amino acid sequence of SEQ ID NO: 5) when the target or subject is a mouse.

Furthermore, in the present invention, the “abnormal iron metabolism” includes not only abnormal iron metabolisms caused by diseases that cause abnormal iron metabolism, but also cases where bone marrow dysfunction is caused by the administration of anticancer agents, radiotherapy, bone marrow transplantation, etc. Moreover, in the present invention, the “abnormal iron metabolism” includes abnormal iron metabolisms mediated by IL-6 (for example, anemia associated with Castleman's disease) and those caused by bone marrow dysfunction. Furthermore, the “diseases that cause abnormal iron metabolism” include diseases with a high active hepcidin level and diseases with a low active hepcidin level. The diseases with a high active hepcidin level include, for example, hemosiderosis, inflammatory diseases, malignant tumors, hepatitis, arteriosclerosis, myocardial infarction, diabetes, rheumatoid arthritis, renal failure, and bone marrow dysfunction, but are not limited thereto. Alternatively, the diseases with a low active hepcidin level include, for example, hereditary hemochromatosis, iron-deficiency anemia, hemolytic anemia, and bleeding in the digestive system (for example, tumor and ulcer of the digestive system, etc), but are not limited thereto.

In the methods of the present invention, the “step of determining the amount of active hepcidin in a serum sample” comprises, for example, the steps of:

(a) mixing a serum sample with a carrier having the property of binding to the active forms of (1) hepcidin-20 and/or (2) hepcidin-25; and

(b) determining the amount of active hepcidin bound to the carrier.

The step described above in (a) is preferably conducted under conditions where the above-described carrier only binds polypeptides whose substantial pI value is 8 or more. For example, a chip with cation exchange groups, such as CM10, may be used. The step can be carried out by contacting the chip with diluted samples at or above pH 8 and then washing the chip with a washing buffer at or above pH 8. The noise in the measurement of active hepcidin can be reduced by adjusting polypeptides with a pI greater than or equal to 8 to have positive charges. This renders more accurate and simpler the diagnosis of abnormal iron metabolism and diseases caused by abnormal iron metabolism, and the selection of optimal agents for the treatment of subjects with such a disease. The preferred pI is 9.2 or greater and a pI of 10 or higher is more preferred.

Such methods include, for example, mass spectrometry. Mass spectrometry includes, for example, SELDI-TOF-MS and MALDI-TOF-TOF. SELDI-TOF-MS is preferred.

SELDI-TOF-MS is a method that comprises immobilizing carriers onto the surface of a chip to be used in a time-of-flight mass spectrometer; contacting test samples with the chip surface with immobilized carriers; washing the chip under an appropriate condition; and determining the mass of proteins trapped onto the chip surface using the time-of-flight mass spectrometer.

SELDI-TOF-MS is an abbreviation of time-of-flight mass spectrography (TOF-MS) using surface-enhanced laser desorption/ionization (SELDI).

Chips with immobilized carriers on their surface are generally referred to as protein chips. In the present invention, protein chips include, for example, chemical chips with chemical properties such as hydrophobic type, cation exchange type, anion exchange type, metal ion type, or normal phase type; activated chips for the analysis of specific binding (interaction); and biological chips of the antibody, receptor, or DNA type. Preferred chips are chemical chips with immobilized cation exchange groups (more preferably CM10), chemical chips with immobilized metal ions (more preferably IMAC30-Cu), or chemical chips with immobilized hydrophobic groups (more preferably H4). CM10 is a chip with immobilized carboxymethyl groups on its surface. IMAC30-Cu is a chip with immobilized NTA on its surface. H4 is a chip that has the same binding property as that of the C16 reverse phase column.

Alternatively, biological chips with immobilized antibodies that bind to hepcidin-20 or hepcidin-25 can also be used in the present invention. In general, antibodies are immobilized onto chips via carbonyldiimidazole or activated epoxy groups.

SELDI-TOF-MS generally comprises steps (1) to (5) described below; however, SELDI-TOF-MS in the present invention is not limited to methods comprising these steps, as long as they allow the detection of active hepcidin.

(1) Step of Adding Serum Samples onto a Protein Chip

A serum sample diluted with a buffer (20 times diluted in this Example) is applied to a protein chip. The chip is incubated (for 30 min in this Example).

(2) Step of Washing the Protein Chip after Sample Application

The chip surface is washed to remove proteins and other molecules that are not bound to the chip surface.

(3) Step of Applying Energy Absorption Molecules (EAMs) to the Protein Chip after Washing

To enhance the ionization of proteins trapped onto the chip surface, energy absorption molecules (EAMs) are injected and the chip is dried. In the present invention, EAM denotes molecules that are capable of absorbing energy from a laser desorption/ionization source and, thereafter, contribute to desorption and ionization of analyte molecules in contact therewith. EAMs of the present invention are not particularly limited, as long as it can be used in the protein chip system. EAMs include, for example, cinnamic acid derivatives, sinapinic acid (SPA), cyanohydroxy cinnamic acid (CHCA) and dihydroxybenzoic acid, ferulic acid, and hydroxyacetophenone derivatives. A preferred EAM is CHCA.

The noise in the measurement of active hepcidin can be reduced by removing polypeptides as much as possible other than polypeptides with pI of 8 or higher, preferably 9.2 or higher, and more preferably 10 or higher, at the time of or prior to the binding of proteins in a serum sample to the surface of the protein chip described above. This renders more accurate and simpler the diagnosis of abnormal iron metabolism and diseases that cause abnormal iron metabolism, and the selection of optimal agents for the treatment of subjects with such a disease. The removal methods described above are not particularly limited. Samples that are suitable for the measurement of active hepcidin can be obtained, for example, by adsorption with chemical chips with immobilized cation exchange groups, such as CM10, at pH 8 to 10.

### (4) Step of Determining Protein Mass and Quantity

Mass numbers of proteins are determined by TOF-MS. When a pulsed UV laser is irradiated onto the protein chip after injection of the EAM, proteins ionized upon receiving the energy are accelerated through a constant voltage. The ionized protein travels toward the ion detector on the opposite side in the vacuum tube. The ion detector translates the detected ion information into the mass vs. charge ratio (mass-to-charge ratio, m/z). The signal intensity is also detected; thus, both protein mass and quantity can be determined. The “protein quantity” is assessed as a peak height on the data output screen. Such measurements can be achieved using ProteinChip (SELDI-TOF-MS, Ciphergen), AXIM-TOF² (MALDI-TOF-TOF, Shimadzu Co.), etc.

### (5) Step of Analyzing Protein Expression

In general, SELDI-TOF-MS uses computers installed with measurement and analysis software. These computers can analyze data and display the number of detected proteins, signal intensities, and determined molecular weights of the detected proteins. Furthermore, the computer can display the data obtained in various formats. In the present invention, standard spectra can be displayed. It is possible to use useful formats, which include Biomarker Wizard (Ciphergen) and Biomarker Patterns® Software (BPS) (Ciphergen). Such analyses can be carried out using ProteinChip (SELDI-TOF-MS, Ciphergen), AXIM-TOF² (MALDI-TOF-TOF, Shimadzu Co.), etc.

The present invention also provides kits that are used in the diagnostic methods described above. The kits include, for example, protein chips and washing solutions to be used in the diagnostic methods described above, instruction manuals, and the like.

Furthermore, the present invention provides agents for the prevention or treatment of diseases that cause abnormal iron metabolism, which are administered to subjects in which active hepcidin in serum is increased or decreased when compared with a subject who is not affected with a disease that causes abnormal iron metabolism.

Whether active hepcidin in serum is increased or decreased when compared with a subject who is not affected with a disease that causes abnormal iron metabolism can be tested using the methods described above.

The agents of the present invention are administered to subjects in which active hepcidin in serum is increased or decreased. Such agents include, for example, erythropoietin, anti-IL-6 agents, and iron preparations, but are not limited thereto.

The administration methods and doses of the agents, their pharmaceutically acceptable carriers (other than the active ingredients), and the like, are known. Such information can be used in the present invention.

The administration methods can be either oral or parenteral. Specifically, parenteral administration methods include injection, nasal administration, pulmonary administration, and percutaneous administration. A pharmaceutical composition of the present invention can be injected locally or systemically; for example, by intravenous injection, intramuscular injection, intraperitoneal injection, or subcutaneous injection. Furthermore, appropriate administration methods can be selected according to the patient's age and symptoms.

The single-administration dose can be selected, for example, from within the range of 0.0001 to 1,000 mg/kg of body weight. Alternatively, the dose can be selected from within the range of 0.001 to 100,000 mg/kg of body weight for each patient; however, the dose of an agent of the present invention is not limited to these examples.

The agents of the present invention can be formulated according to conventional methods (see, for example, Remington's Pharmaceutical Science, latest edition, Mark Publishing Company, Easton, U.S.A), and may comprise pharmaceutically acceptable carriers and additives. Such carriers include, for example, surfactants, excipients, coloring agents, flavoring agents, preservatives, stabilizers, buffering agents, suspending agents, isotonizing agents, binders, disintegrators, lubricants, fluidity promoters, and corrigents, but are not limited thereto. It is also possible to use other appropriate conventional carriers. Specifically, such carriers include light anhydrous silicic acid, lactose, crystalline cellulose, mannitol, starch, carmellose calcium, carmellose sodium, hydroxypropylcellulose, hydroxypropylmethylcellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, and inorganic salts.

In addition, the present invention provides compositions that are used in the methods of the present invention, which comprise polypeptides whose substantial pI is 8 or more and which are purified from blood collected from a subject. The noise in the measurement of active hepcidin can be reduced by using a “composition that comprises polypeptides whose substantial pI is 8 or more”. This renders more accurate and simpler the diagnosis of abnormal iron metabolism and diseases that cause abnormal iron metabolism, and the selection of optimal agents for the treatment of subjects with such a disease. Such compositions can produce a desired effect, as long as they comprise polypeptides with a pI greater than or equal to 8. The preferred pI is 9.2 or greater and a pI of 10 or higher is more preferred.

All prior-art documents cited in the specification are incorporated herein by reference.

### EXAMPLES

The present invention is specifically described below with reference to the Examples; however, it is not to be construed as being limited thereto.

### Example 1

### 1 Materials and Methods

### 1.1 Study Participants

Serum samples were obtained from 40 hemodialysis patients before and after hemodialysis. All patients were receiving hemodialysis treatment three times a week. In addition, serum samples were collected from patients with acute pyelonephritis (two patients), pneumonia (two patients), and sepsis (two patients), as well as from 16 healthy volunteers with normal renal function. Serial samples of serum and urine obtained from two patients with acute pyelonephritis and two hemodialysis patients who had received intravenous iron administration for iron deficiency were also analyzed. Red blood cell (RBC) count, hemoglobin (Hb), hematocrit (Ht), plasma iron, and ferritin were evaluated for the clinical assessment of iron status and anemia. Serum samples were separated by centrifugation (3,000 rpm) and stored at −80° C. until assayed. Informed consent for the analysis of the protein profiling of serum and/or urine was obtained from all the participants.

### 1.2 SELDI-TOF-MS Analysis of Serum and Urine Proteins

For preliminary trials, multiple types of protein chips were used with various surface characteristics, which included weak cation exchange, strong anion exchange, and immobilized metal affinity capture for protein molecules that bind divalent cationic copper. The present inventor eventually chose the mobilized metal affinity capture ProteinChip® array (IMAC 30-Cu) for the entire study because of its reproducibility in detecting protein species from serum and urine. SELDI analysis was performed according to the manufacturer's manual, with some modifications. In brief, serum and urine samples were diluted 20-fold and 10-fold, respectively, with binding buffer (phosphate buffered saline, pH 7.4). Using a bioprocessor, 40 μl of diluted samples were applied onto different arrays of an IMAC 30-Cu chip pretreated with 100 mM copper sulfate binding buffer and the chip was incubated at room temperature for 30 min with constant horizontal shaking. For confirmation of the reproducibility, each sample was applied to two separate spots on an IMAC 30-Cu chip. Unbound proteins were removed by washing three times with binding buffer for 5 min. The arrays were rinsed twice with 400 μl of water, were air-dried, and 0.5 μl of alpha-cyano-4-hydroxy-cinnamic acid (CHCA; Ciphergen) in 50% acetonitrile and 0.5% trifluoroacetic acid was added twice onto the surface of the chip. Next, the arrays were analyzed using the Ciphergen ProteinChip® Reader PBS II. The proteins with CHCA were ionized and their molecular masses were determined by TOF analysis. The mass-to-charge ratio (m/z) of each of the proteins/peptides captured on the array surface was determined according to externally calibrated standards: Arg8-vasopressin (1,084.25 Da), somatostatin (1,637.9 Da), dynorphin (2,147.5 Da), bovine insulin beta-chain (3,495.94 Da), human insulin (5,807.65 Da), bovine ubiquitin (8,564.8 Da), and bovine cytochrome C (12,230.9 Da). The mass spectra of the samples were generated using an average of 80 laser shots at a laser intensity of 180 and a detector sensitivity of 10. Synthetic hepcidin-25 (Peptide Institute Inc, Osaka) was used as a marker of 2,789 Da. The peak intensity data were normalized with total ion current using Biomarker Wizard (Ciphergen ProteinChip® Software 3.1.1) to compensate for the variations in the concentration of samples loaded onto a spot.

### 1.3 pI Value of Peptides

The pI value was evaluated on the cationic ProteinChip® array (CM 10). Six aliquots of one serum sample were diluted with Tris/HCl buffer in the pH range of 8 to 10, at 0.4 intervals.

### 1.4 Reduction by Dithiothreitol (DTT) and Alkylation by Iodoacetamide

Samples were reduced with 5 mM DTT (25 mM ammonium carbonate) for 2 min at 70° C. to detect the number of internal disulfide bridges. DTT reduction added two hydrogen atoms to form two free sulfhydryl groups for each disulfide bond broken; thus, the molecular weight increased by 2 Da. Furthermore, alkylation (room temperature, 30 min) using 50 mM iodoacetamide (25 mM ammonium carbonate) increased the molecular weight by 114 Da.

1.5 Separation and Purification of Serum 2,789 m/z from Dialysis Patient Serum

The serum was adsorbed onto a CM ceramic hyper DF spin column at pH 8. After washing, the column was eluted with 500 mM NaCl to obtain a crude preparation of 2,789 m/z. The extract was further fractionated by reverse phase HPLC and the purified 2,789 m/z was confirmed using a protein chip. The purified 2,789 m/z was immobilized onto the protein chip, the chip was loaded onto an interface, and its amino acid sequence was analyzed by CID tandem MS. The amino acid sequence was searched using Mascot.

### 1.6 IL-6 Concentration

The concentrations of serum IL-6 were assayed using enzyme-linked immunosorbent assay (ELISA) kits (R&D, Quantikine HS, Minneapolis, Minn., USA), according to the manufacturer's protocol.

### 1.7 Statistical Analyses

Statistical analysis of paired variables was performed using the parametric paired t test. Pearson correlation coefficients were calculated to study the linear relationship between continuous variables. P values were calculated based on a two-sided alternative hypothesis and were considered significant at P<0.05.

### 2 Results

### 2.1 Proteins Related to Iron Metabolism

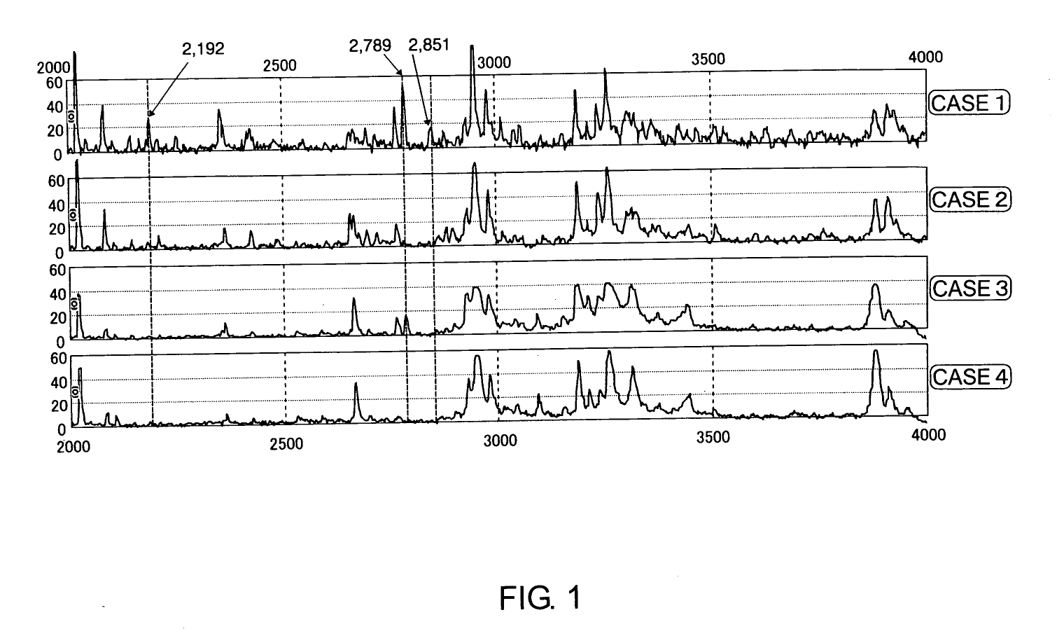

FIG. 1 shows representative protein mass spectra of sera from two hemodialysis patients and two volunteers with normal renal function in an m/z range of 2,000 to 4,000, which revealed differences in their peak patterns. From the spectra of the 62 participants, the Biomarker Wizard software automatically detected 114 peaks in the range of 1,000 to 15,000 m/z.

To identify the proteins related to iron metabolism, the correlation coefficients between the intensity of each peak and RBC, Hb, Ht, serum iron, or ferritin were calculated (Table 1).

TABLE 1

| | Ferritin | Fe | RBC | Hb | Ht |
|---|---|---|---|---|---|
| m/z | r | r | r | r | r |
| 2192 | 0.93 | 0.37 | −0.25 | −0.06 | −0.11 |
| 2789 | 0.83 | 0.41 | −0.28 | −0.09 | −0.15 |
| 2851 | 0.80 | 0.36 | −0.20 | −0.04 | −0.05 |
| 1497 | 0.65 | 0.18 | −0.12 | −0.06 | −0.05 |
| 1347 | 0.60 | 0.15 | −0.21 | −0.14 | −0.15 |
| 1038 | 0.46 | 0.00 | −0.02 | −0.13 | −0.10 |
| 2366 | 0.42 | 0.23 | 0.03 | −0.04 | 0.03 |
| 2085 | 0.38 | 0.06 | 0.21 | 0.23 | 0.28 |
| 1531 | 0.35 | 0.16 | 0.22 | 0.14 | 0.23 |
| 4973 | 0.31 | −0.01 | −0.08 | −0.17 | −0.09 |

r, Pearson correlation coefficient

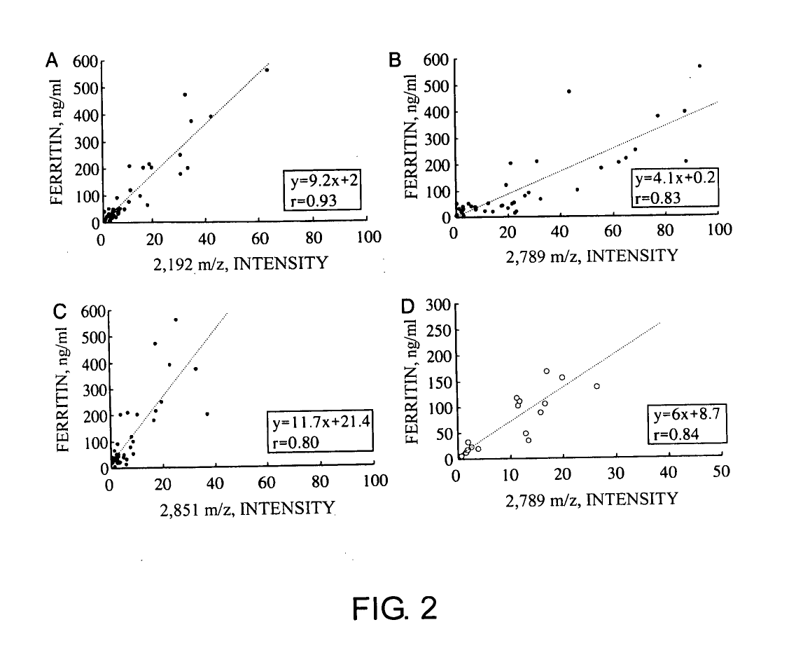

For serum ferritin, the intensities of only 3 of the 114 peaks (2,192, 2,789, and 2,851 m/z) had good correlation coefficients, i.e., greater than 0.8 (r=0.93, 0.83, and 0.80, respectively) (FIGS. 2A, B, and C). No peaks had a significant correlation with either RBC, Hb, Ht, or serum iron. Correlations of the intensities of these peaks with serum ferritin were also observed in volunteers with normal renal function (FIG. 2D). The levels of the 2,789 m/z peak in hemodialysis patients were higher than those in healthy volunteers with the same levels of ferritin (FIG. 2B, D). A significant correlation was also observed between the intensities of the serum 2,192 and 2,789 m/z peaks (r=0.9), which suggests that these two molecules are regulated by the same mechanism.

By searching through a protein database (SWISS PROT), the present inventor found that the peaks of 2,192 m/z and 2,789 m/z closely corresponded to the molecular weight of human hepcidin-20 (theoretical molecular weight in reduced form: 2,199.78 Da) and hepcidin-25 (2,797.41 Da), respectively. In a previous report (Park C H, Valore E V, Waring A J, Ganz T. J Biol. Chem. 2001; 276:7806-7810), the molecular weight of hepcidin-20 and hepcidin-25, as assessed by MALDI, was 2,191.77 and 2,789.40 Da, respectively. The peak at 2,851 m/z was not observed on the database.

### 2.2 Characteristic pI Evaluation of the Serum 2,192 and 2,789 m/z Peaks

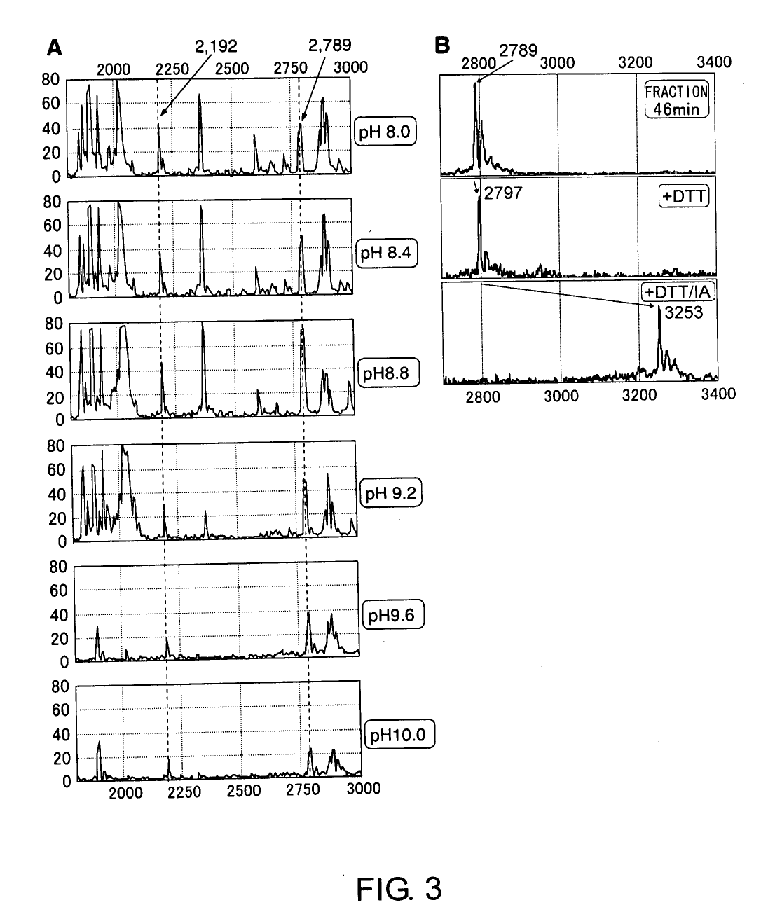

The cationic protein chip CM 10 was used for the pI evaluation of the 2,192 and 2,789 m/z peaks. Tris/HCl in the pH range of 8.0 to 10.0 was used as the diluting and washing buffer. Both peptides still bound to the chip at pH 10 (FIG. 3A), which suggests that they were strong cationic peptides.

### 2.3 Separation and Purification of the Serum 2,789 m/z Peptide

The serum 2,789 m/z peptide was separated and purified from a dialysis patient serum. The 2,789 m/z peptide was bound to the cation protein chip CM 10 at pH 8 and was eluted with 500 mM NaCl. Under similar conditions, a crude preparation of 2,789 m/z was obtained using a CM ceramic hyper DF spin column. The preparation was further fractionated by reverse phase HPLC. The purified 2,789 m/z peptide was confirmed using a protein chip (FIG. 3B, top).

### 2.4 Characteristic Disulfide Bridge Evaluation of the Purified 2,789 m/z Peak

The isolated and purified serum peak at 2,789 m/z (FIG. 3B, top) was reduced by DTT treatment and appeared as a new peak at 2,797 m/z (FIG. 3B, middle), which indicates that the peptide gained 8 Da of molecular weight after reduction. A new 3,253 m/z peak appeared after alkylation by iodoacetamide treatment, which indicates that the molecular weight increased by 456 Da (FIG. 3B, bottom). These results suggest that the peptide at 2,879 m/z contained four internal disulfide bonds.

### 2.5 Identification of the Purified 2,789 m/z Peak

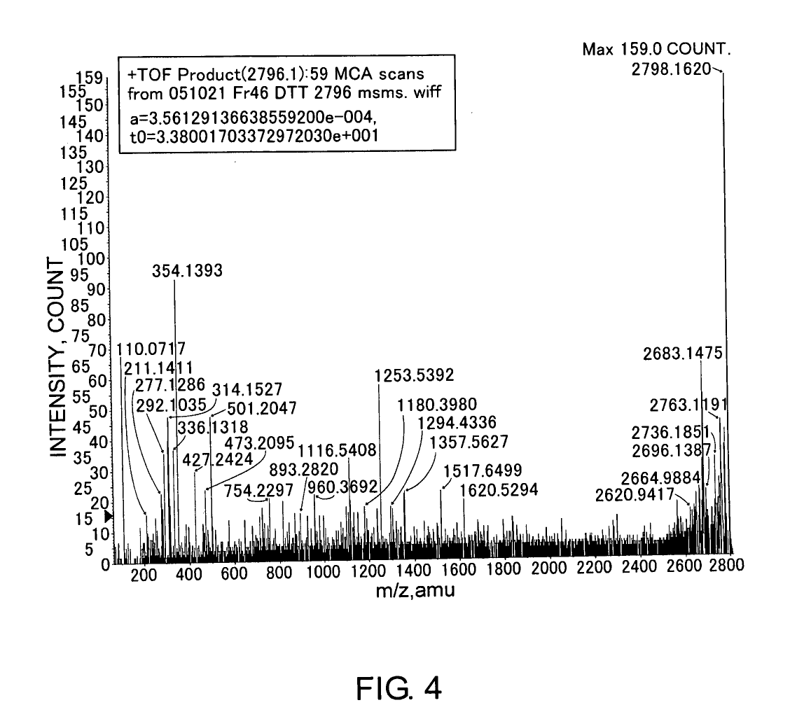

The purified 2,789 m/z peptide was immobilized onto a protein chip and analyzed by CID tandem MS. The amino acid sequence of the produced ions (FIG. 4), identified by Mascot search, was identical to that of hepcidin-25.

### 2.6 Semi-Quantitative Measurement Method for Hepcidin-25

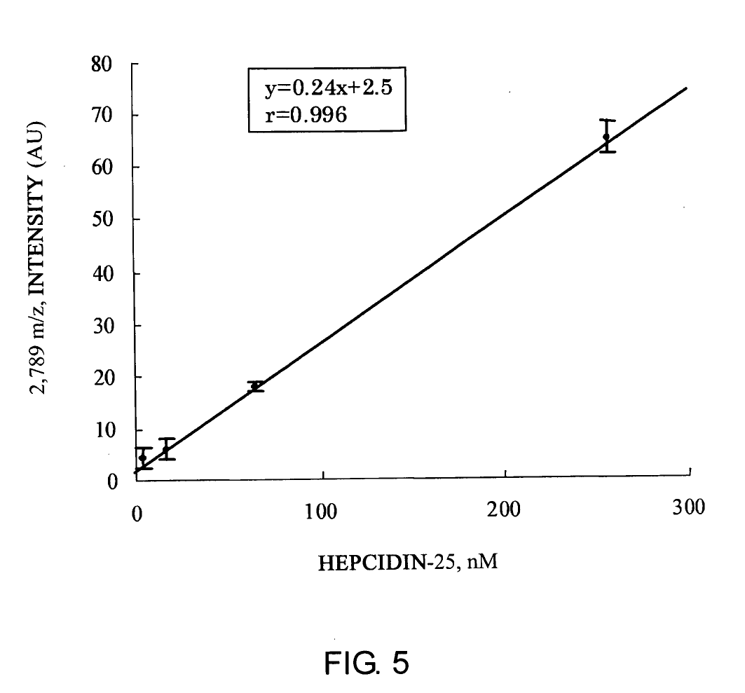

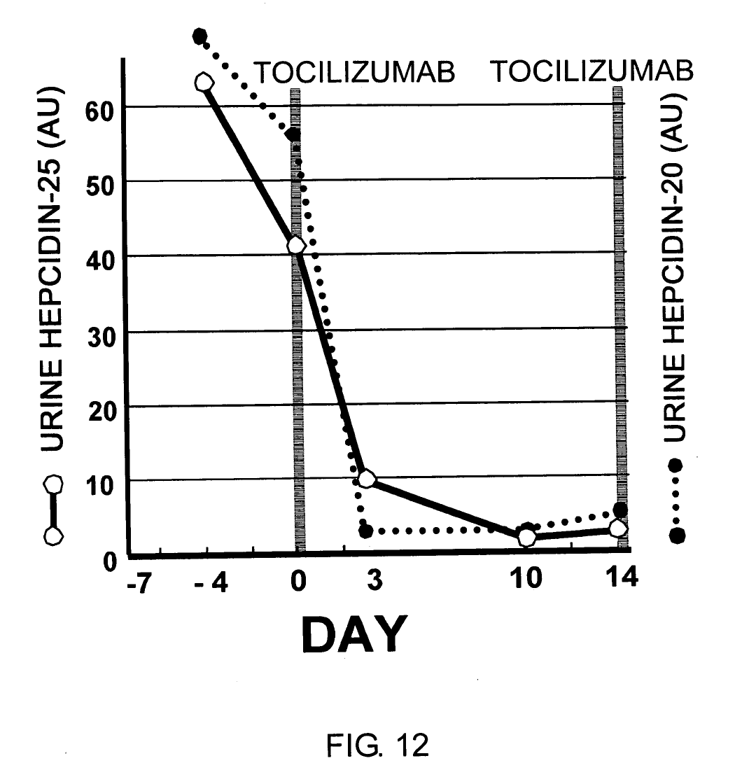

Synthetic hepcidin-25 was added at 4, 16, 64, or 256 nM to serum without 2,789 m/z. A measurement using a protein chip showed that the correlation was r=0.9 while the CV values were 20 to 30% (FIG. 5). This suggests the clinical applicability of this method as a semi-quantitative measurement method (FIG. 12). The intensity for hepcidin-25 was 30 AU or less in healthy volunteers.

### 2.7 Clearance of the Peaks of the Three Peptides by Hemodialysis

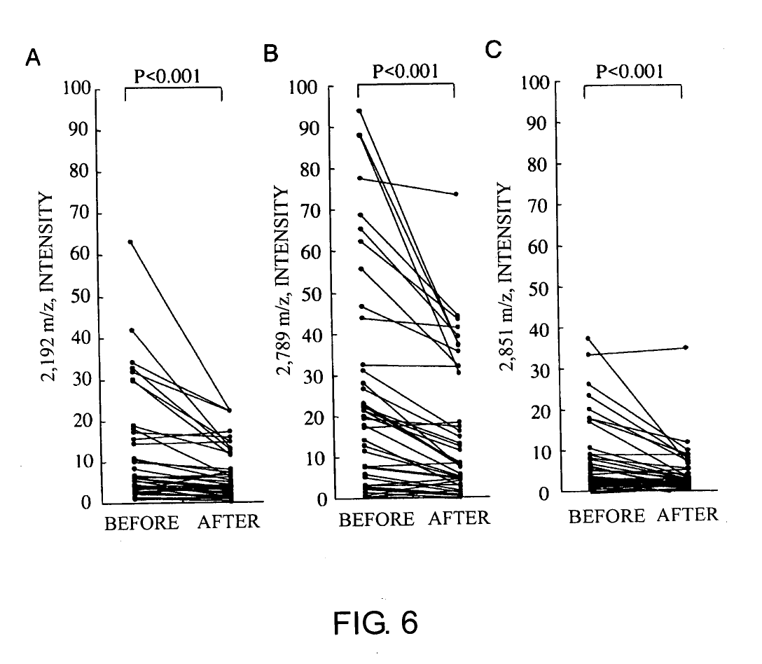

FIG. 6 shows the changes in the levels of the 2,192, 2,789, and 2,851 m/z peaks before and after hemodialysis. In comparison with the removal rate of blood urea nitrogen measured at the same time, which was 71.4%±2.4% in these patients, the removal rates of the 2,192, 2,789, and 2,851 m/z peptides by hemodialysis (45±19%, 46±23%, and 52±30%, respectively) were not satisfactory, despite their small size.

2.8 Time Course of the Peak at 2,789 m/z after Iron Loading

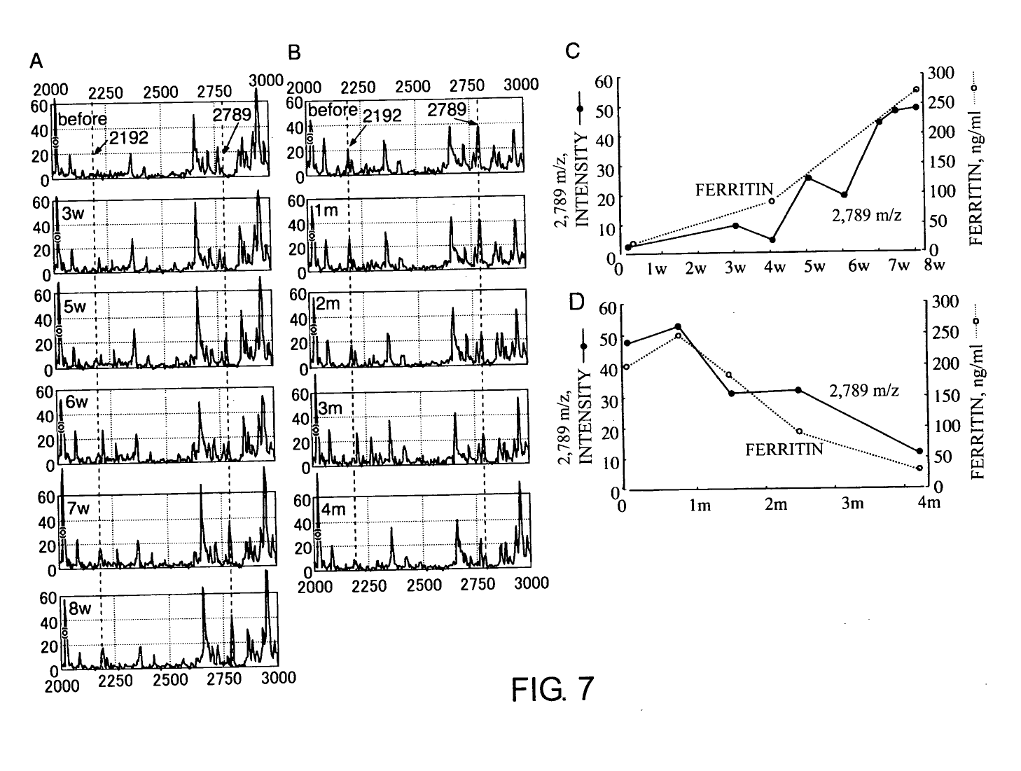

FIGS. 7A and C shows the serial profiling of the serum from a 72-year-old woman who was intravenously administered 40 mg of iron twice a week for eight weeks, for severe anemia. The intensity of the peak at 2,789 m/z increased gradually because of iron administration and the levels of serum ferritin increased from 11.3 to 273 ng/ml. In contrast, the levels of ferritin dropped from 202 to 28 ng/ml over a period of four months in a 34-year-old woman who suffered from bleeding due to menstruation (FIGS. 7B and D) and the intensity of the peak at 2,789 m/z also diminished gradually. In both cases, no remarkable changes were found in the peak patterns, with the exception of the peaks at 2,192 and 2,789 m/z. The dialysis conditions for these two cases were the same during these periods.

2.9 Serial Analysis of Serum and Urine from a Patient with Acute Pyelonephritis

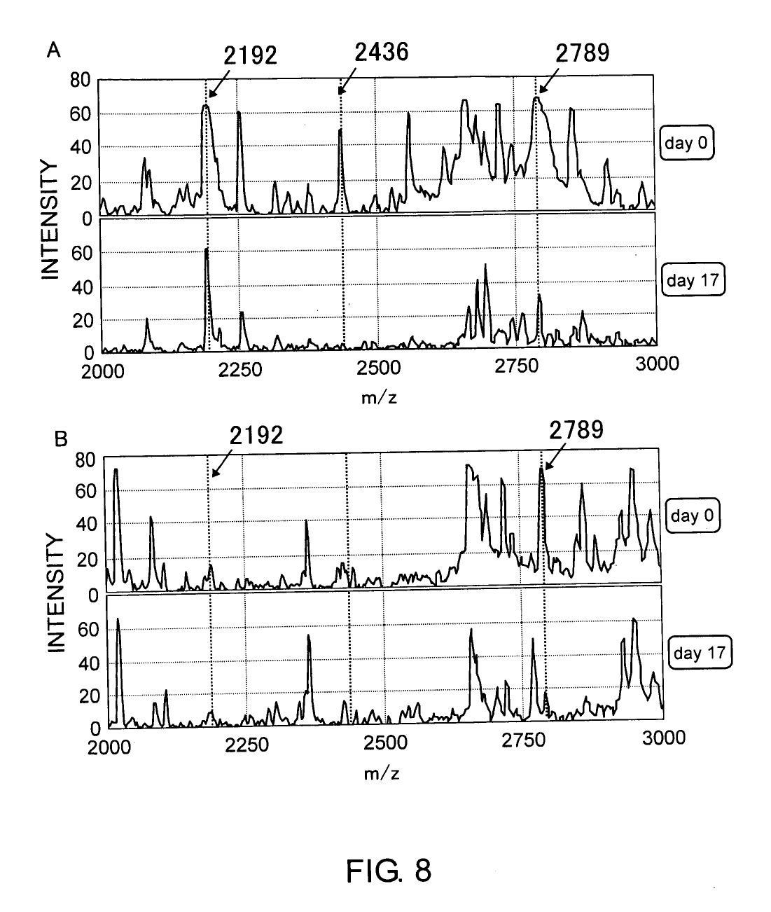

The protein profiling of the serum and urine from a patient with acute pyelonephritis is shown in FIG. 8. The patient was treated with antibiotics. In both serum and urine samples, the peaks at 2,192 and 2,789 m/z, which were detected before the treatment, diminished gradually. The levels of ferritin on day 0 and day 17 were 343.7 and 230.6 ng/ml, respectively.

2.10 Association of the Intensities of the Peaks at 2,192 and 2,789 m/z with Serum IL-6 Concentration

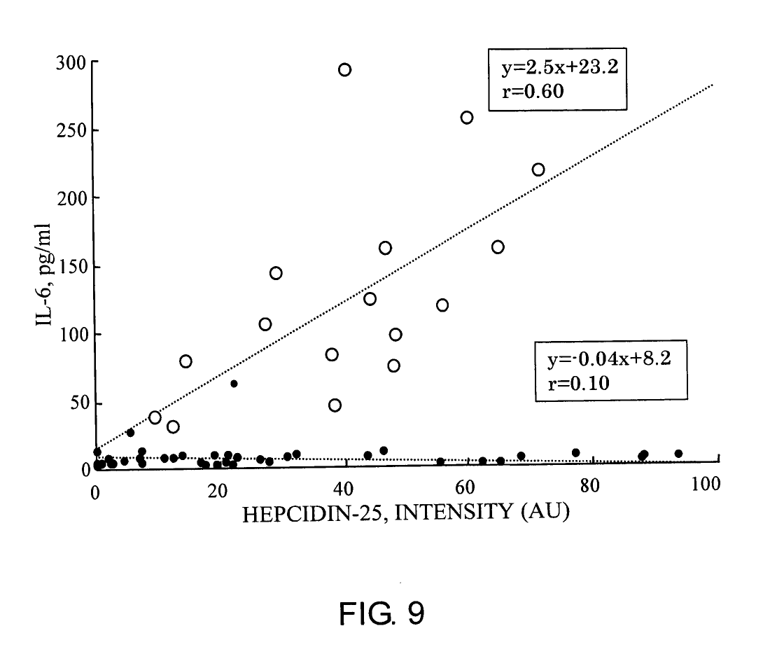

The concentration of serum IL-6 was lower than 20 pg/ml in all hemodialysis patients, with the exception of one patient with systemic lupus erythematosus (SLE) who had been treated with a glucocorticoid (FIG. 9). The levels of serum IL-6 in patients with acute pyelonephritis, SLE, pneumonia, and sepsis had a good correlation with the intensity of the 2,789 m/z peak (r=0.6).

### 3 Discussion

In this Example, the present inventor employed the SELDI technology to identify serum proteins that are regulated by body iron status and found a cluster of peptides at 2,192, 2,789, and 2,851 m/z, based on the correlation with the level of serum ferritin. The sizes of two of these peptides (2,192 and 2,789 m/z) matched almost exactly with those of hepcidin-20 and -25, which were reported by Park et al. (Park C H, Valore E V, Waring A J, Ganz T. J Biol. Chem. 2001; 276:7806-7810). Furthermore, these peptides were strongly cationic and had four internal disulfide bridges; these characteristics were consistent with those of the active forms of hepcidin; therefore, the present inventor concluded that the two peptides at 2,192 and 2,789 m/z were hepcidin-20 and -25, respectively. The peak intensities of these two peptides increased due to iron loading and inflammation. In addition, the present inventor identified another peptide at 2,851 m/z, the peak intensity of which showed a clear correlation with the level of serum ferritin in hemodialysis patients.

Since hepcidin is a key molecule for iron homeostasis, the measurement of its serum concentration could be a useful tool for the differential diagnosis of anemia and iron overload in human diseases (Ganz T. Best Pract Res Clin Haematol. 2005; 18:171-182). The serum concentration of prohepcidin, but not of bioactive hepcidin, is measured by ELISA (Taes Y E, Wuyts B, Boelaert J R, De Vriese A S, Delanghe J R. Clin Chem Lab Med. 2004; 42:387-389; Kulaksiz H, Gehrke S G, Janetzko A, et al. Gut. 2004 May; 53(5):735-743); however, to date, a reliable and practical method for quantification of the bioactive forms of serum hepcidin has not been developed, probably because of the difficulty involved in the development of specific antibodies against hepcidin-20, -22, and -25. Based on MALDI-TOF mass analysis, Park et al. (Park C H, Valore E V, Waring A J, Ganz T. J Biol. Chem. 2001; 276:7806-7810) reported that hepcidin-20, -22, and -25 are the major forms in urine. Recently, Kemna et al. (Kemna E, Tjalsma H, Laarakkers C, Nemeth E, Willems H, Swinkels D. Blood. 2005 Jul. 19; [Epub ahead of print]) reported a semi-quantitative method to analyze urine hepcidin using SELDI technology with hydrophilic normal phase chips (NP20). The present inventor analyzed urine samples from patients with iron deficiency anemia, hereditary hemochromatosis, and sepsis and detected three peaks that corresponded to the molecular weights of hepcidin-20, -22, and -25; however, they did not detect the bioactive forms of hepcidin in serum using this method.

In the present Example, the present inventor detected peaks corresponding to hepcidin-20 and hepcidin-25 in serum. Prohepcidin, which is produced in the liver, needs to be processed by specific proteases to generate the bioactive forms of hepcidin that are observed in the circulation (Park C H, Valore E V, Waring A J, Ganz T. J Biol. Chem. 2001; 276:7806-7810); therefore, the data obtained from serum analysis is very important for further understanding of iron metabolism. Although Nemeth et al. (Nemeth E, Rivera S, Gabayan V, et al. J Clin Invest. 2004; 113:1271-1276) have shown that the stimulatory effects of dietary iron rapidly induce the expression of hepcidin and rapidly diminish iron absorption from the intestine, the kinetics of hepcidin in the serum during this “mucosal block phenomenon” remains unknown. The SELDI technology would be useful to analyze the rapid kinetics of hepcidin in serum.

In previous studies (Taes Y E, Wuyts B, Boelaert J R, De Vriese A S, Delanghe J R. Clin Chem Lab Med. 2004; 42:387-389; Kulaksiz H, Gehrke S G, Janetzko A, et al. Gut. 2004 May; 53(5):735-743; Dallalio G, Fleury T, Means R T. Br J. Haematol. 2003; 122:996-1000), elevated levels of serum prohepcidin measured by ELISA were found in renal insufficiency; however, there was no relationship between serum prohepcidin concentration and body iron status. In the present study, the levels of the two peaks corresponding to hepcidin-20 and -25 in hemodialysis patients were also higher than those in healthy volunteers with normal renal function who had the same levels of serum ferritin. In dialysis patients, only 46% of the 2,789 m/z peptide (hepcidin-25) was removed by hemodialysis, despite its small size, resulting in its accumulation in the serum; however, the serum levels of this peptide were highly correlated with the concentration of serum ferritin. This correlation was also observed in serial samples after iron loading in a patient under the same dialysis conditions. Nemeth et al. (Nemeth E, Valore E V, Territo M, Schiller G, Lichtenstein A, Ganz T. Blood. 2003; 101:2461-2463) also reported that urinary hepcidin excretion correlated well with the serum ferritin concentration in anemia of inflammation and sepsis. These findings suggest that the expression of hepcidin in serum may be regulated by the body iron status, in combination with ferritin. The toxicity of iron in the cellular system is largely attributable to its capacity to participate in the generation of reactive oxygen species and ferritin captures and buffers the intracellular labile iron pool to protect cells (Torti F M, Torti S V. Blood. 2002; 99:3505-3516). On the other hand, hepcidin may prevent iron toxicity by inhibiting iron absorption from the intestine; thus, in the condition of iron overload, active hepcidin may, in combination with ferritin, play a key physiological role along in the maintenance of iron homeostasis.

At present, it is well known that, in addition to iron loading, hepcidin is induced by anemia, hypoxia, and inflammation (Nicolas G, Chauvet C, Viatte L, et al. J Clin Invest. 2002; 110:1037-1044) and that all of these stimuli also induce the expression of ferritin (Torti F M, Torti S V. Blood. 2002; 99:3505-3516). The association between hepcidin and inflammation is becoming clearer (Lee P, Peng H, Gelbart T, Wang L, Beutler E. Proc Natl Acad Sci USA. 2005; 102:1906-1910). IL-6 appears to be a major cytokine for the induction of hepcidin production in inflammation because in the model of inflammation induced by turpentine, the expression of hepcidin mRNA in the liver is increased in wild-type mice, but not in IL-6 knockout mice (Nemeth E, Rivera S, Gabayan V, et al. J Clin Invest. 2004; 113:1271-1276). Only two human studies have reported that the urinary excretion of hepcidin is greatly increased in patients with anemia of inflammation, acute infection with epididymitis (Nemeth E, Valore E V, Territo M, Schiller G, Lichtenstein A, Ganz T. Blood. 2003; 101:2461-2463), and severe sepsis (Kemna E, Tjalsma H, Laarakkers C, Nemeth E, Willems H, Swinkels D. Blood. 2005 Jul. 19; [Epub ahead of print]). Nemeth et al. (Nemeth E, Rivera S, Gabayan V, et al. J Clin Invest. 2004; 113:1271-1276) reported that IL-6 infusion in human healthy volunteers with normal renal function caused a rapid increase in hepcidin excretion in urine; however, no clinical data on the correlation between hepcidin and IL-6 have been reported to date. In this Example, the increased serum levels of IL-6 in acute pyelonephritis, pneumonia, and sepsis were accompanied by increased serum levels of both ferritin and hepcidin. These findings imply that IL-6 may cause an increase in both ferritin and hepcidin in human diseases. Rogers (Rogers J T. Blood. 1996; 87:2525-2537) reported that IL-6 induces the expression of ferritin at the translational level, as the levels of the mRNAs for the ferritin H-subunits and L-subunits remain unchanged, whereas ferritin subunit synthesis increases. In the case of hepcidin, IL-6 induces hepcidin expression at the mRNA level (Nemeth E, Valore E V, Territo M, Schiller G, Lichtenstein A, Ganz T. Blood. 2003; 101:2461-2463); however, the mechanism of induction of hepcidin by iron overload remains unclear (Ganz T. Best Pract Res Clin Haematol. 2005; 18:171-182).

Low-molecular-weight proteins/peptides comprise several classes of physiologically important factors, which include cytokines, chemokines, peptide hormones, and proteolytic fragments of lager proteins (Tirumalai R S, Chan K C, Prieto D A, Issaq H J, Conrads T P, Veenstra T D. Mol Cell Proteomics. 2003; 2:1096-1103). Interestingly, 114 small proteins with a molecular weight lower than 15,000 Da were detectable in the serum, even in the volunteers with normal renal function, although these peptides are smaller than the presumed cutoff size for kidney filtration. Furthermore, the two peptides at 2,192 and 2,789 m/z (hepcidin-20 and -25, respectively) were sufficiently cationic to be filtered through the glomerular basement membrane, which is anionic. This suggests that these peptides may be part of large protein complex or may have unknown specific retention mechanisms. In acute pyelonephritis, these two peptides were detected not only in serum but also in urine; however, it is unclear whether urinary hepcidin is filtered from the blood or originates from the kidney. Hepcidin is released from the kidneys, at least partly, as it is expressed in the renal tubular cells (Kulaksiz H, Theilig F, Bachmann S, et al. J. Endocrinol. 2005; 184:361-370). An infection in the renal interstitium could activate the prohepcidin produced by the renal tubular cells.

In conclusion, the SELDI results indicated that the 2,192 and 2,789 m/z are hepcidin-20 and -25, respectively. These peptides partially accumulated with declining renal function, but the concentration of these peptides in the serum was well correlated with body iron status, which was represented by the levels of serum ferritin. These peptides were also induced during urinary infection. Data of the present inventor demonstrate that SELDI is a useful tool to detect and semi-quantify the bioactive forms of hepcidin in both serum and urine.

### Example 2

Multicentric Castleman's disease (MCD) is a rare lymphoproliferative disorder that manifests various systemic symptoms, which include lymphadenopathy, fever, and microcytic anemia. Elevation of the serum level of interleukin-6 (IL-6) is one of its common features and the anti-IL-6 receptor antibody tocilizumab (registered trademark: Actemra) has been used to alleviate the symptoms of MCD. The etiology of the microcytic anemia observed in MCD has been implicated in the upregulation of hepcidin, which is a key regulator of iron metabolism, and IL-6 is known to stimulate production of hepcidin in the liver.

The present inventor monitored serum and urine hepcidin before and after tocilizumab treatment in two Castleman's disease cases who began treatment with tocilizumab (registered trademark: Actemra), from 7 days before treatment to 14 days after the start of the treatment. The measurement of hepcidin was performed in the manner described in Example 1.

TABLE 2
Before treatment

| | |
|---|---|
| Case 1 | Case 2 |
| 24 yo F | 32 yo F |

| | | | | |
|---|---|---|---|---|
| Hb | 4.5 | g/dl | 9.8 | g/dl |
| MCV | 69 | fL | 82 | fL |
| CRP | 28.9 | mg/dl | 14.6 | mg/dl |
| Fe | 9 | mg/dl | 39 | mg/dl |
| UIBC | 146 | mg/dl | 123 | mg/dl |
| Ferritin | 151 | ng/ml | 327 | ng/ml |
| IL-6 | 215 | pg/ml | 15 | pg/ml (<4 pg/ml) |
| VEGF | 1,121 | pg/ml | 1,364 | pg/ml |

Laboratory findings before treatment in two Castleman's disease cases

In both cases, downregulation of serum hepcidin-25 was observed within a single day after the administration of the first dose of tocilizumab and prior to the improvement of microcytic anemia and the decrease of both CRP and serum ferritin levels. This result indicates that expression of hepcidin is controlled by the IL-6 pathway in a very quick manner in clinical situations.

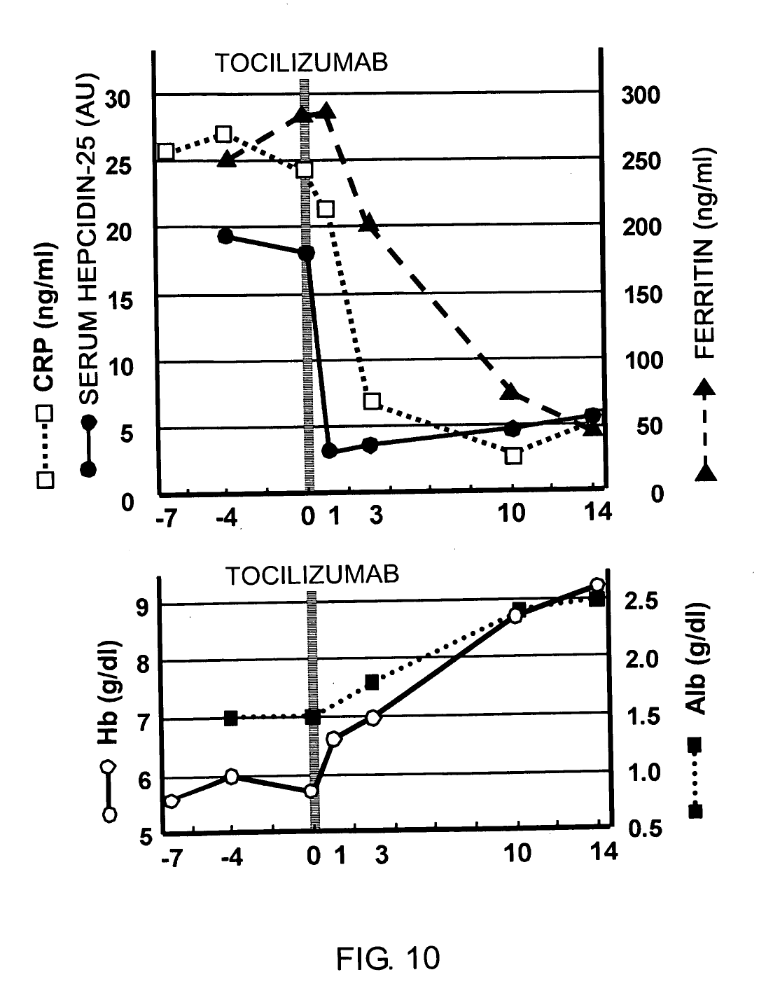

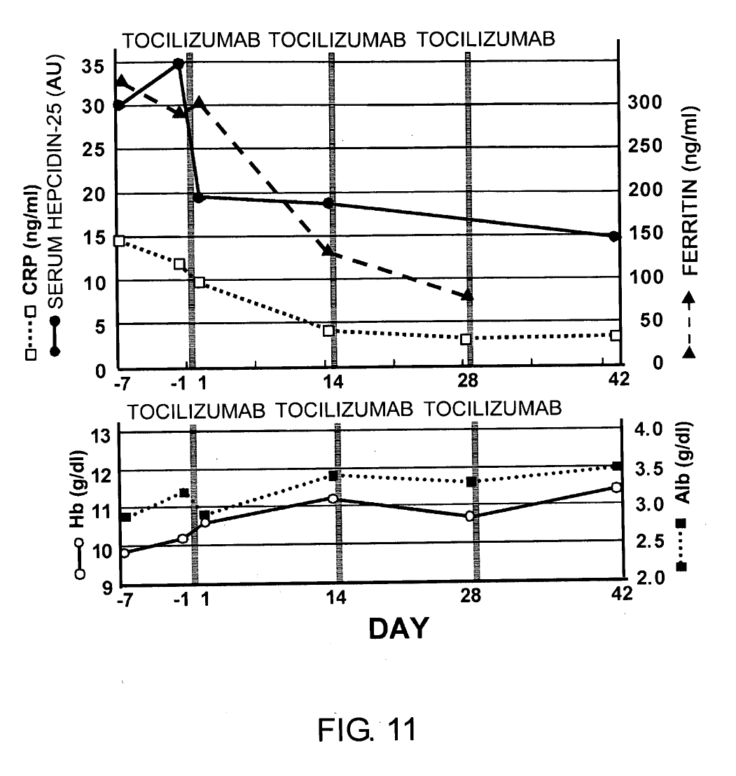

More specifically, the treatment alleviated the high CRP levels, anemia, and hypoalbuminemia, while the serum ferritin level was reduced slowly (case 1, FIG. 10; case 2, FIG. 11). The serum hepcidin-25 level was drastically reduced on the day following the first tocilizumab administration. This preceded the decrease in CRP and ferritin levels. Urine samples were also analyzed in case 1 (FIG. 12). The urine levels of both hepcidin-20 and hepcidin-25 were dramatically reduced on day 3. The drop in hepcidin level after tocilizumab administration, which had preceded the decrease in the serum ferritin level, supports the hypothesis that IL-6 regulates iron metabolism via hepcidin, and also shows that the half-life of hepcidin in serum is short; thus, tocilizumab is assumed to be useful in alleviating not only Castleman's disease but also anemia accompanied by the IL-6-mediated increase in hepcidin level and abnormal iron metabolism. Furthermore, the hepcidin measurement system using mass spectrometry is assumed to be useful in assessment of the therapeutic effect of tocilizumab in Castleman's disease.

### Example 3

Hepcidin is a liver-produced peptide that regulates iron metabolism by decreasing iron absorption from the intestine and by blocking its release from iron stores; however, the precise mechanisms of regulation of hepcidin production have yet to be elucidated. Several investigators have recently speculated that suppression of hepcidin production during erythropoiesis is not directly caused by anemia, hypoxia, or erythropoietin, but by an unknown erythropoietic factor. As bone marrow function is completely suppressed by conditioning chemotherapy during hematopoietic stem cell transplantation (SCT), it should be meaningful to assess the serum level of hepcidin before and after SCT.

The present inventor monitored the serum level of hepcidin, interleukin-6 (IL-6), and other serum factors involved in iron metabolism from one week before (W −1) to four weeks after (W+4) the day of SCT in ten cases of autologous and allogeneic SCT for leukemia. The serum level of hepcidin-25, which is the major form of active hepcidin peptide, was measured semi-quantitatively using SELDI-TOF mass spectrometry.

TABLE 3
Patient characteristics

| | | | | | | | Pyrexia | G- |
|---|---|---|---|---|---|---|---|---|
| Patient | Disease | Status | Conditioning | SCT | Engraftment | Infection | (day) | CSF |
| 1 | PTCL | CR | Flu/Mel/TBI4 | mini CBT | 14 | + | 1 | + |
| 2 | DLBCL | CR | R/MEAM | auto PBSCT | 11 | + | 14 | + |
| 3 | MDS/AML | CR | Flu/Bu/TBI4 | mini BMT | \textcircled{26} | + | 5 | − |
| 4 | AML | CR | Flu/Mel/TBI4 | full UBMT | \textcircled{39} | − | 3 | + |
| 5 | T-LBL | CR | TBI12/CY | auto PBSCT | 12 | + | 4 | − |
| 6 | ATL | CR | Flu/Bu/TBI4 | mini UBMT | \textcircled{32} | + | 6 | + |
| 7 | DLBCL | CR | R-MEAM | auto PBSCT | 12 | + | 9 | − |
| 8 | AML | CR | TBI12/CY | full RBMT | 21 | + | 15 | − |
| 9 | ALL | CR | TBI12/CY | full RBMT | 19 | + | 9 | + |
| 10 | MDS/AML | PR | Flu/Mel/TBI | mini CBT | \textcircled{25} | + | 7 | + |

Data on bone marrow transplantation of leukemia patients

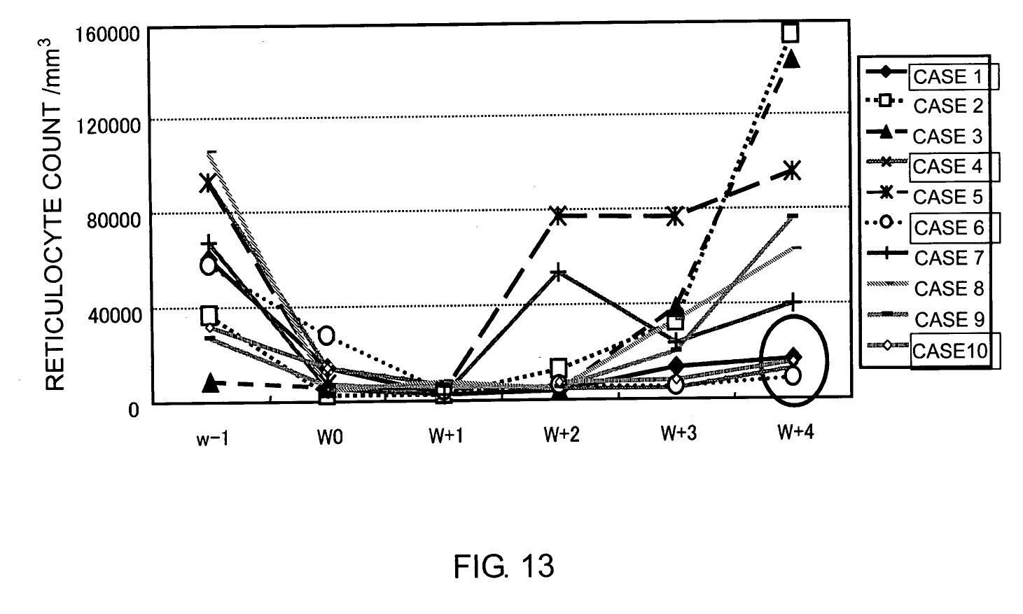

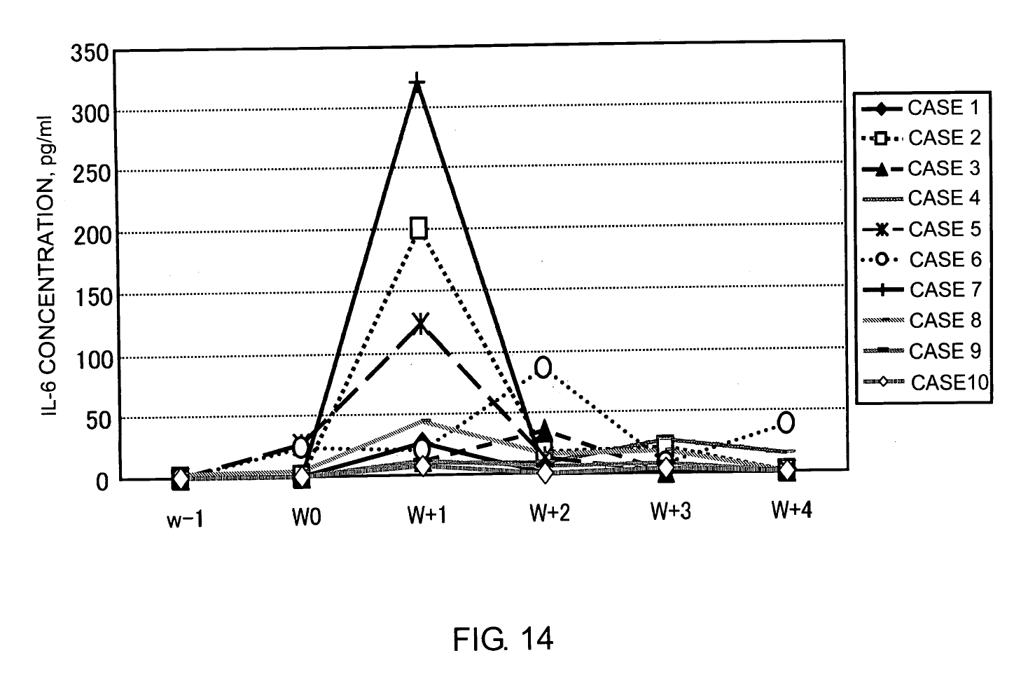

The therapeutic pretreatment starting one week before SCT (W −1) reduced the reticulocyte count in weeks 0 and +1; thus, bone marrow function was almost completely suppressed. The reticulocyte count started to recover in week +2; however, the recovery was delayed in cases 1, 4, 6, and 10 (FIG. 13). The serum IL-6 level reached a peak in week +1 in almost all cases and then decreased, which suggests that the SCT treatment had a significant influence (FIG. 14).

The level of hepcidin at week −1 before therapeutic pretreatment was already high when compared with the normal range (mean±SD, 65.4±41.6 AU), and it further increased after SCT (FIG. 14). The peak level of hepcidin was observed at week +1 (167.1±67 AU) in eight cases, and the other two cases showed peaks at week 0 (153 AU) and week +2 (267 AU). The hepcidin level gradually decreased with the reticulocyte engraftment, but the level tended to be high in cases 1, 4, 6, and 10, in whom recovery of hematopoiesis had been delayed. This implies that expression of hepcidin correlates with the suppression of bone marrow function indicated by reticulocyte count. Furthermore, hepcidin was already high before therapeutic pretreatment, when IL-6 was normal, and even in a case without any elevation of serum IL-6, a clear peak of serum hepcidin-25 was observed at week +1. These results indicate that the serum hepcidin level during SCT may be upregulated partly by IL-6 and also by other hematopoietic factors, which are increased at an early stage after SCT.

### INDUSTRIAL APPLICABILITY

The present invention provides novel methods for the diagnosis of abnormal iron metabolism. The diagnostic methods of the present invention can directly measure the active hepcidin in blood alone, and thus enable direct assessment of hepcidin in a state of prebinding to the iron transporter ferroportin. Accordingly, the methods of the present invention can provide a more clinically relevant diagnosis for abnormal iron metabolism when compared with the prior art. Furthermore, the present invention enables real-time examination of abnormal iron metabolism changes, which makes it possible, for example, to diagnose diseases that cause abnormal iron metabolism, to determine the administration timing and dose of an agent for the treatment of such a disease, and to assess the hematopoietic function of the bone marrow (for example, the hematopoietic function after radiotherapy, bone marrow transplantation, or administration of agents, e.g., anticancer agents, that impair bone marrow function because of their adverse effects). In addition, the present invention enables the assessment of the effect of the administration of therapeutic agents for the treatment of diseases that cause abnormal iron metabolism, e.g., anti-inflammatory agents that target IL-1 and IL-6; thus, the present invention is advantageous in that it enables selection of optimal agents for the treatment of the diseases.

1. A method for diagnosing an abnormal iron metabolism, which comprises determining the amount of active hepcidin in a serum sample prepared from blood collected from a subject and comparing the amount of active hepcidin in the serum sample from the patient to a control, wherein an increased or decreased amount of active hepcidin in the serum sample from the patient as compared to the control indicates an abnormal iron metabolism in the patient.

2. A method for diagnosing a disease that causes an abnormal iron metabolism, which comprises determining the amount of active hepcidin in a serum sample prepared from blood collected from a subject and comparing the amount of active hepcidin in the serum sample collected from the subject to a control, wherein an increased or decreased amount of active hepcidin in the serum sample from the subject as compared to the control indicates the disease causes an abnormal iron metabolism.

3. A method for selecting an optimal agent for treating a subject affected with a disease that causes an abnormal iron metabolism, which comprises the steps of:
(a) determining the amount of active hepcidin in a serum sample prepared from blood collected from a subject administered with a test agent, wherein the subject is affected with a disease that causes an abnormal iron metabolism;
(b) processing, in the same way as in step (a), each of one or more test agents different from the test agent used in step (a);
(c) comparing the amount of each active hepcidin determined in step (a) and step (b) with that in a control serum sample; and
(d) selecting a test agent that brings the amount of active hepcidin closest to that in the control serum sample, based on the comparison result obtained in step (c).

4. A method for determining the timing to administer an agent used to treat a disease which causes an abnormal iron metabolism, which comprises the step of determining the amount of active hepcidin in each serum sample prepared from blood collected from a subject over time.

5. A method for assessing restoration of bone marrow function, which comprises determining the amount of active hepcidin in each serum sample prepared from blood collected over time from a subject treated by radiotherapy or bone marrow transplantation.

6. The method of claim 1, wherein the abnormal iron metabolism is mediated by IL-6.

7. The method of claim 6, wherein the IL-6-mediated abnormal iron metabolism is anemia in Castleman's disease.

8. The method of claim 1, wherein the abnormal iron metabolism is caused by a bone marrow dysfunction.

9. The method of claim 1, wherein the active hepcidin is hepcidin-20 and/or hepcidin-25.

10. The method of claim 1, wherein the step of determining the amount of active hepcidin in a serum sample comprises the steps of:
(a) mixing a serum sample with a carrier that has the property of binding to the active forms of (1) hepcidin-20 and/or (2) hepcidin-25; and
(b) determining the amount of active hepcidin bound to the carrier.

11. The method of claim 10, wherein step (a) is conducted under conditions where only a polypeptide with a substantial pI value of 8 or more binds to the carrier.

12. The method of claim 1, wherein the active hepcidin is detected by SELDI-TOF-MS.

13. A kit to be used in the method of claim 1.

14. An agent that is used to treat or prevent a disease that causes an abnormal iron metabolism wherein the agent is administered to a subject in whom the amount of active hepcidin in serum is increased or decreased when compared with a subject without a disease that causes the abnormal iron metabolism.

15. An active hepcidin polypeptide, wherein said active hepcidin has a substantial pI value of 8 or more and is purified from blood collected from a subject.


SEQUENCE LISTING (5 sequences)

SEQ ID NO: 1 - protein, 20 aa, Homo sapiens
ICIFCCGCCHRSKCGMCCKT

SEQ ID NO: 2 - protein, 25 aa, Homo sapiens
DTHFPICIFCCGCCHRSKCGMCCKT

SEQ ID NO: 3 - protein, 25 aa, Rattus norvegicus
DTNFPICLFCCKCCKNSSCGLCCIT

SEQ ID NO: 4 - protein, 25 aa, Mus musculus
DTNFPICIFCCKCCNNSQCGICCKT

SEQ ID NO: 5 - protein, 25 aa, Mus musculus
DINFPICRFCCQCCNKPSCGICCEE